(19) United States
(12) Patent Application Publication
(10) Pub. No.: US 2011/0275649 A1
(43) Pub. Date: Nov. 10, 2011

(54) Combination therapy for the prevention of statin induced diabetes

(75) Inventor: Wayne H. Kaesemeyer, Chapel Hill, NC (US)

(73) Assignee: Palmetto Pharmaceuticals LLC, Charleston, SC (US)

(21) Appl. No.: 13/067,016

(22) Filed: May 3, 2011

Related U.S. Application Data
(60) Provisional application No. 61/343,839, filed on May 5, 2010.

(51) Int. Cl. A61K 31/495; A61K 31/22; A61K 31/404; A61K 31/40; A61K 31/505; B65B 3/04; A61K 31/496; A61K 31/4458; A61K 31/195; A61K 31/336; A61K 31/19; A61P 3/10; A61K 31/366; A61K 31/47

(52) U.S. Cl. 514/254.03; 514/460; 514/510; 514/419; 514/423; 514/275; 514/311; 514/252.12; 514/317; 514/565; 514/475; 514/557; 53/473

(57) ABSTRACT

Novel combinations comprising HMG CoA reductase inhibitors, or statins, with partial fatty acid oxidation inhibitors (pFOXi), and methods for their use, are disclosed. These combinations are useful in preventing or reducing the risk of developing diabetes which results from therapy with a statin.

### CROSS-REFERENCE TO RELATED APPLICATIONS

This application claims benefit under 35 U.S.C. §119 to U.S. Provisional Application No. 61/343,839, filed May 5, 2010, the contents of which are incorporated by reference herein.

### BACKGROUND OF THE INVENTION

Beginning with the introduction of lovastatin in 1987, HMG-CoA reductase inhibitors, or statins, have become the most frequently prescribed class of drugs. The ability of statins to reduce morbidity and mortality rates in patients with cardiovascular disease involves inhibition of HMG CoA reductase, the enzyme responsible for production of mevalonate and the rate-limiting step in the synthesis of cholesterol. On the other hand, there are benefits from statin therapy in patients with cardiovascular disease that are unrelated to cholesterol lowering and the inhibition of HMG CoA reductase. And preliminary studies suggest benefits from statins may extend to conditions other than cardiovascular diseases, including neurologic, rheumatologic and other classes of diseases. Therefore, the term “statin therapy” refers to any condition or disease treated with a statin or in need of treatment with a statin.

Very recently it has become apparent that despite their well established benefits in preventing and treating cardiovascular diseases, statins increase the risk of diabetes. This came to attention early on in the recent JUPITER trial in which there was an increase in diabetes in the group receiving rosuvastatin. More recently a meta-analysis of over 90,000 patients enrolled in 13 major clinical trials revealed a 9% incidence in those newly initiated to statin therapy. The mechanism whereby statins induce this problem is not yet fully understood but appears to be related to statin lipid solubility with atorvastatin and simvastatin, the two most lipid soluble statins, being much more likely to induce diabetes than pravastatin which is water soluble. Statins may unfavorably affect insulin secretion and exacerbate insulin resistance. This may involve adipocyte metabolism. Insulin resistance has been shown to be increased in adipocytes exposed to a statin. In addition, statins have a hypolipidemic effect as a result of promoting free fatty acid utilization over glucose in hepatocytes. This would be another cause of increased insulin resistance. There may also be effects by statins on key mitochondrial substrate regulating enzymes such as 3-ketoacyl-CoA-thiolase, malonyl CoA decarboxylase and pyruvate dehydrogenase that increase insulin resistance as a result of shifting mitochondrial substrate metabolism to free fatty acid utilization. This remains to be investigated.

Also, it has recently been observed that pFOX inhibitors (pFOXi), drugs generally used in the treatment of angina, have additional benefits on glucose metabolism in angina patients with diabetes. The exact mechanism for this effect is unknown but most likely results from a shift of substrate for energy production from free fatty acids to glucose. This preferential shift to increase glucose utilization in the production of ATP by the mitochondrial electron transport chain is thought to increase sensitivity to insulin and reduce glucose and glycosylated hemoglobin levels along with the development of diabetes, especially type 2 diabetes.

Kaesemeyer, published Patent Application 2006/0205727 discloses a combination of a HMG CoA reductase inhibitor, such as statin, and pFOX inhibitors for the treatment of ischemic end-stage complications of endothelial dysfunction, such as acute coronary syndrome (ACS), chronic angina, and diabetes, especially in type II diabetics. Further, the application is about treatment, making statins antianginal agents by adding a pFOX to their formulation to form a simple fixed-dose combination with very narrow dose ranges of both components of the combination. The Kaesemeyer application does not teach that the combination is useful for preventing or reducing the risk of developing diabetes in subjects newly initiated to statin therapy and induced by the therapy itself, a condition that was not clinically recognized until very recently. And, the application teaches only fixed doses of each component of the combination on the basis of results of new long term clinical outcomes trials and does not teach varying doses of the statin and pFOX inhibitor components in the combination so as to treat conditions for which statin indications are already approved. Finally, the application does not disclose formulating the combination in a dual delivery system that permits immediate release of the statin and sustained release of the pFOX inhibitor which enables once daily dosing and simplifies approval of the formulations by regulatory agencies.

### SUMMARY OF THE INVENTION

The present invention relates to the newly recognized problem of statin induced diabetes. The present invention relates to the prevention of diabetes, new onset diabetes to be specific, and not to the treatment of end stage ischemic complications of diabetes, which is an angina equivalent condition. The present invention concerns a unit dose combination comprising HMG CoA reductase, such as statin and pFOX inhibitor to prevent or reduce the risk of developing diabetes in statin therapy. The present invention relates to a unit dose combination of HMG CoA reductase, such as statin, and pFOX in a novel delivery system that would permit once daily dosing of the combination while preserving current and most desirable release profiles for each component of the combination. In the present invention, the HMG CoA reductase can be a statin. The present invention further relates to a unit dose combination comprising statin that is specially formulated so that it does not increase a subject's risk of developing statin induced diabetes. The present invention also relates to a unit dose combination comprising statin that will reduce the risk of diabetes during the course of a statin therapy.

The present invention relates to a method of preventing diabetes resulting from a statin therapy or reducing the risk of diabetes resulting from the statin therapy in a subject who is in need of the statin therapy comprising administering a unit dose combination of HMG-CoA reductase inhibitor and pFOX inhibitor to the subject in an effective therapeutic amount to prevent diabetes resulting from the statin therapy or to reduce the risk of diabetes resulting from the statin therapy. The subject, who is in need of the statin therapy, newly initiates the statin therapy. The HMG-CoA reductase inhibitor can be a statin. The statin can be selected from simvastatin, lovastatin, pravastatin, compactin, fluvastatin, dalvastatin, atorvastatin, rosuvastatin, pitavastatin, or mixtures thereof.

The present invention relates to using simvastatin as the statin. The present invention relates to using lovastatin as the statin. The present invention relates to using pravastatin as the statin. The present invention relates to using fluvastatin as the statin. The present invention relates to using dalvastatin as the statin. The present invention relates to using compactin as the statin. The present invention relates to using atorvastatin as the statin. The present invention relates to using rosuvastatin as the statin. The present invention relates to using pitavastatin as the statin.

The present invention relates to using a pFOX inhibitor selected from trimetazidine, ranolazine, CVT 4325, perhexiline, mildronate, etomoxir, dichloroacetate, a pyruvate dehydrogenase agonist, a malonyl CoA decarboxylase inhibitor, or mixtures thereof

The present invention relates to combining the statin and pFOX inhibitor in a single tablet, blister pack, administration kit, IV solution, transdermal patch, rectal suppository, inhalational device for immediate, sustained-release or a combination of immediate-release and sustained-release dosing.

An aspect of the present invention relates to combining the HMG-CoA reductase inhibitor and pFOX inhibitor in a single tablet with a dual delivery system which immediately releases the statin and sustained-releases the pFOX inhibitor. In another aspect of the invention, the statin is atorvastatin and the pFOX inhibitor is trimetazidine being combined in a single tablet with a dual delivery system which immediately releases the statin and sustained-releases the pFOX inhibitor. In yet another aspect of the invention, the statin is rosuvastatin and the pFOX inhibitor is trimetazidine being combined in a single tablet with a dual delivery system which immediately releases the statin and sustained-releases the pFOX inhibitor. In further aspect of the invention, the statin is pitavastatin and the pFOX inhibitor is trimetazidine being combined in a single tablet with a dual delivery system which immediately releases the statin and sustained-releases the pFOX inhibitor. In another further aspect of the invention, the statin is pitavastatin and the pFOX inhibitor is ranolazine being combined in a single tablet with a dual delivery system which immediately releases the statin and sustained-releases the pFOX inhibitor.

Regarding Kaesemeyer, published Patent Application 2006/0205727 and the present invention, there are four aspects to diabetes and statins and the combinations described herein:

1) preventing diabetes or reducing the risk of diabetes occurring as a result of a subject receiving a statin, regardless of the indication for the statin, the subject being naive to therapy with the statin;

2) preventing diabetes or reducing the risk of diabetes occurring as a result of the natural history of the disease unrelated to its treatment;

3) treating established diabetes, i.e., by lowering glucose levels, etc., the disease itself, regardless of its cause; and

4) treating the ischemic complications of established or end-stage diabetes unrelated to preventing the occurrence of diabetes or treating the condition itself, as described in aspect 3 above.

The present invention concerns aspect 1 above. The previous Kaesemeyer application and combinations described therein concern aspect 4 above. Neither the previous Kaesemeyer application nor the present invention is concerned with preventing naturally occurring diabetes or diabetes treatment, as described in aspects 2 and 3 above.

In view of these recent developments, the embodiments of this disclosure provide for a combination of a HMG CoA reductase inhibitor, such as a statin, and pFOXi in an amount effective for preventing or reducing the risk of developing new-onset diabetes from treatment with a statin in a subject previously naive to statin therapy.

### DETAILED DESCRIPTION OF THE INVENTION

Below are definitions for terms related to this invention. In describing and claiming the present invention, the following terminology will be used in accordance with the definitions set out below. Notwithstanding, other terminology and definitions may also be found throughout this disclosure as well.

As used in the present invention, preventing the development of diabetes means that diabetes does not occur. As indicated in the discussion, below, of the JUPITER study of nonhyperlipidemic patients taking a statin as compared to nonhyperlipidemic patients taking a placebo, a statistically significant number of patients during the course of a statin therapy developed diabetes.

As used in the present invention, reducing the risk of developing, or preventing, diabetes means a reduction in the number of new physician diagnosed cases of diabetes seen with a statin-pFOXi combination versus a statin used alone in those individuals who are newly initiated on therapy with a statin.

The present invention relates to the recently recognized risk of diabetes, principally type 2 diabetes, which results from the use of statins, seen principally in the treatment of dyslipidemia according to guidelines set forth by the National Cholesterol Education Program (NCEP), but also in the treatment of nonhyperlipidemic individuals at increased risk of diabetes from statins when they are used to prevent cardiovascular diseases by targeting individuals with increased inflammatory biomarkers, such as pulmonary hypertension, or individuals treated with statins for reasons unrelated to cardiovascular diseases such as, but not limited to, rheumatoid arthritis, neurodegenerative disorders and sepsis. It has been observed that pFOX inhibitors have actions that reduce insulin resistance and the risk for the development of diabetes by increasing cellular glucose metabolism that are in addition to their usual anti-ischemic actions. The present invention concerns a combination of HMG CoA reductase, such as statin with pFOX in a unit dose form. The statin is dosed currently without concern for limitations from effects on blood pressure or heart rate, i.e., no hemodynamic or chronotropic effects over those seen with statin alone administration. The present invention contemplates the use of dual drug delivery system that permits delivery of drugs from separate compartments in a single tablet with separate and independent pharmacokinetic profiles for each drug.

Statins are the most frequently prescribed drugs in history. The number of individuals who are newly initiated to statin therapy annually is therefore also very large. A recent large meta-analysis of over 90,000 subjects in 13 clinical trials revealed a 9% risk of diabetes from the use of statins following initiation of statin therapy. In one of those 13 trials, JUPITER, a study involving 17802 nonhyperlipidemic subjects at increased risk of cardiovascular events based on elevated C-reactive protein (CRP) levels, there were 258 cases of physician reported new cases of diabetes in the statin group (2.8%) versus 205 cases in the placebo group (2.3%)[hazard ratio 1.27 (1.5, 1.53), p=0.015]. Therefore the total number of individuals annually at risk of developing diabetes from statin therapy is considered to be very large. So, despite the fact that statins are very effective in treating the ischemic complications of diabetes, in some individuals they also induce the very disease that they treat.

The exact mechanism by which statins induce diabetes is unknown. Preliminary studies suggest that statins affect adipocyte metabolism differently according to their state of cellular differentiation. Furthermore, the risk of diabetes from statins may vary according to a subject's baseline state of obesity.

In so far as diabetes itself, and principally type 2 diabetes, is considered an epidemic with 26 million cases in the US alone, numbers that are anticipated to double over the next ten years due to an ongoing epidemic of obesity, having a statin which itself does not increase the risk of diabetes is extremely desirable. In fact, one has to take notice of the current diabetes epidemic and its association timewise with a similar increase in those being newly initiated on statin therapy and ask if the two trends are causally related.

### Statins

There are a number of statins that are available and approved for use. These include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin. The preferred statins include atorvastatin, pitavastatin, pravastatin, fluvastatin, lovastatin, simvastatin and rosuvastatin. The statin compounds are administered in regimens and at dosages known in the art.

For instance, Fluvastatin sodium, marketed by Novartis Pharmaceuticals as Lescol™, is recommended for a 20-80 mg daily oral dose range, preferably between 20 and 40 mg/day for the majority of patients. 20 to 40 mg daily doses are preferably taken once daily at bedtime. 80 mg daily doses is prescribed as 40 mg doses b.i.d. and recommended only for those individuals in whom the 40 mg daily dose is inadequate to lower LDL levels satisfactorily.

Atorvastatin, offered by Parke Davis as Lipitor™, has a recommended starting daily dose of 10 mg once daily, with an overall daily dose range of from 10 to 80 mg.

Simvastatin, marketed by Merck & Co., Inc., may be administered with a starting dose of 20 mg once a day in the evening, or a 10 mg dose per day for those requiring only a moderate reduction in LDL levels. The recommended overall daily dosage range taken as a single evening dose is from 5 to 80 mg.

Pravastatin sodium, sold as Pravachol™. by Bristol-Meyers Squibb, has a recommended starting dose of 10 or 20 mg per day, taken daily as a single dose at bedtime, with a final overall daily range of from 10 to 40 mg.

Lovastatin, sold by Merck & Co. as Mevacor™, has a recommended daily starting dosage of 20 mg per day taken with the evening meal. The recommended final daily dosage range is from 10 to 80 mg per day in single or divided doses.

Pitavastatin, the most recently approved drug in this class, is administered in a dose range of between 1-4 mg per day.

In general, all of these statins are formulated in a simple delivery system which involves immediate-release kinetic profiles.

pFOX Inhibitors

A “pFOX inhibitor” is any compound that shifts myocardial substrate utilization from free fatty acid to glucose, regardless of the enzyme involved, 3-ketoacyl-CoA-thiolase, malonyl CoA decarboxylase or pyruvate dehydrogenase agonist. A pFOX inhibitor, most preferably one which does not prolong QT intervals, can be used in combination with a HMG CoA reductase inhibitor, commonly referred to as “statins”, in the same patients receiving statins currently without concern for additional major side effects over those seen now with statins used alone. Most notable, the combination does not affect blood pressure or heart rate.

The piperazine derivatives trimetazidine and ranolazine are examples of pFOX inhibitors whose mechanism of action involves shifting ATP production away from fatty acid oxidation in favor of glucose oxidation. Inhibition of fatty acid oxidation results in a reduction in the inhibition of pyruvate dehydrogenase and an increase in glucose oxidation. The amount of oxygen required to phosphorylate a given amount of ATP is greater during fatty acid oxidation than during carbohydrate oxidation. Thus, increasing glucose oxidation reduces oxygen demand without decreasing the ability of tissue to do work.

With trimetazidine, there is also a considerable amount of glucose substrate shifting which occurs as a result of its inhibition of the enzyme 3-keto-CoA-thiolase, or 3-KAT. Trimetazidine has also been shown to: (1) reduce the levels of plasma C-reactive protein in the course of acute myocardial infarction treated with streptokinase and intravenous trimetazidine infusion; (2) hive a beneficial effect in patients with circulatory deficiency through the improvement of hemostatic and biochemical parameters; (3) induce functional improvement in patients with dilated cardiomyopathy via significant improvement of left ventricular function and; (4) improve insulin sensitivity and reduces levels of glycosylated hemoglobin and glucose in diabetics with coronary disease.

Clinical results also suggest that the inflammatory response was limited in patients treated with trimetzidine.

Trimetazidine can be used in a dose range from about 1 to about 2000 mg, preferably in a dose range from about 120 to about 1200 mg and more preferably in a dose range from about 60 to about 600 mg, and most preferably in a dose range from about 30 to about 300 mg. Trimetazidine is formulated as both an immediate-release preparation, with a dose of 20 mg three times daily, and an extended-release preparation of 35 mg twice daily. The preferred delivery system for its administration is extended-release.

Ranolazine, unlike trimetazidine, inhibits the late sodium channel and this is thought to protect myocytes against ischemia. Ranolazine may also result in an increase in insulin secretion by the pancreas which would be beneficial in metabolic syndrome and diabetes. However, ranolazine is not preferred because it causes QT interval prolongation and undergoes metabolism via the CYP3A4 system in the liver and is therefore prone to drug-drug interactions which further aggravate QT interval prolongation. Furthermore, its daily dose, 500-1500 mg twice daily, is much higher than trimetazidine's. This would make it much more difficult to formulate it into a single tablet with a statin for once daily administration; however, QT prolongation did not cause excess arrythmogenic events in Merlin—TIMI 36 and a combination of pitavastatin with ranolazine is a possibility due to the low 1-4 mg/ day dose range of pitavastatin. That is, one canformulate a single tablet with pitavastatin at 1-4mg with 1000 mg of ranolazine in the delivery system described below and dose the tablet on a daily basis. To achieve a current total approved ranolazine dose of 2000 mg one would need to give 2 tablets with the lower 1-mg strengths of pitavastin as guided by the subject's requirement for pitavastatin. Finally, it should be noted that pitavastatin does not undergo metabolism by the 3A4 system so it would be safer than those statins that are metabolized by 3A4 when used in combination with ranolazine.

Ranolazine is currently formulated in an extended-release delivery system. Early studies with it formulated as an immediate-release preparation were unsuccessful. Its dose range in studies has been between 500 to 1500 mg dosed twice daily. Due to side effects at the highest dose, the approved dose is limited to 1000 mg twice daily. Other suitable pFOX inhibitors include perhexiline maleate, mildronate, CVT 4325, malonyl CoA carboxylase inhibitors, etomoxir and dichloroacetate.

Perhexiline maleate is an anti-anginal agent. Its mechanism of action as an anti-anginal agent has not been fully elucidated in humans; however, in vitro studies suggest that perhexiline causes inhibition of myocardial fatty acid catabolism (e.g. by inhibition of carnitine palmitoyltransferase-1: CPT-1) with a concomitant increase in glucose utilization and consequent oxygen-sparing effect. This is likely to have two consequences:
(i) increased myocardial efficiency, and decreased potential for impairment of myocardial function during ischemia, and
(ii) increase insulin sensitivity and reduce the risk of developing type 2 diabetes.

Delivery System

In general any unit dose combination of a statin with a pFOX could be entertained. This would include a single tablet, blister pack, administration kit, intravenous solution, transdermal patch, rectal suppository and inhalation device.

An aspect of this invention relates to a dual drug delivery system in which a single pill is administered once daily and comprises a statin that has an immediate-release kinetic profile, typical of the statins currently prescribed above, combined with a pFOX inhibitor that has a separate once daily sustained-release kinetic profile. Any type of dual drug delivery system may be used in the present invention. A typical type of delivery system is the Geminex Dual Delivery System marketed by Penwest Pharmaceuticals or the Geomatrix System of SkyePharma. For example, Geminex™ is a bilayered dual-delivery tablet technology that incorporates both an immediate-release and one or two controlled-release components and can deliver drugs or isomers with two different and unique release profiles at two different rates.

The advantage of this dual drug delivery system is that it permits statin administration by an immediate release kinetic profile, the way statins are currently formulated and dosed, while administering the pFOX inhibitors with a sustained release profile which is the current way they are administered. Therefore, the approval of the combination would need only bioequivalence testing for the statin component.

### EXAMPLE 1

Unit dose combinations of atorvastatin and trimetazidine are prepared in accordance with the present invention. Immediate-release formulations of atorvastatin 10, 20, 40, and 80 mg are combined in a dual immediate-release extended-release delivery system as described above with an extended release core of trimetazidine. Trimetazidine is used in a dose range from about 1 to about 2000 mg, preferably in a dose range from about 120 to about 1200 mg and more preferably in a dose range from about 60 to about 600 mg, and most preferably in a dose range from about 30 to about 300 mg. Accordingly, the following unit dose combinations of atorvastatin and trimetazidine are prepared:
Atorvastatin 10 mg and an extended-release preparation of 60 mg trimetazidine;
Atorvastatin 20 mg and an extended-release preparation of 60 mg trimetazidine;
Atorvastatin 40 mg and an extended-release preparation of 60 mg trimetazidine;
Atorvastatin 80 mg and an extended-release preparation of 60 mg trimetazidine;
Atorvastatin 10 mg and an extended-release preparation of 120 mg trimetazidine;
Atorvastatin 20 mg and an extended-release preparation of 120 mg trimetazidine;
Atorvastatin 40 mg and an extended-release preparation of 120 mg trimetazidine;
Atorvastatin 80 mg and an extended-release preparation of 120 mg trimetazidine;
Atorvastatin 10 mg and an extended-release preparation of 240 mg trimetazidine;
Atorvastatin 20 mg and an extended-release preparation of 240 mg trimetazidine;
Atorvastatin 40 mg and an extended-release preparation of 240 mg trimetazidine; and
Atorvastatin 80 mg and an extended-release preparation of 240 mg trimetazidine.

The above unit dose combinations can be used in place of the current tablet of Lipitor™ without the risk of diabetes recently reported. It is also expected that there will be no new hemodyamic, chronotropic or other adverse effects limiting the use of the unit dose combinations.

It should be noted that the atorvastatin component of this tablet could be replaced by similar doses of other statins such as lovastatin, simvastatin, pravastatin, rosuvastatin and fluvastatin, all currently approved statins except for pitavastatin whose dose range is different and described in example 2

### EXAMPLE 2

Unit dose combinations of pitavastatin and ranolazine are prepared in accordance with the present invention. Immediate-release formulations of 1, 2, 3 and 4 mg of pitavastatin are combined in a dual immediate-release extended-release delivery system as described above with an extended release core of ranolazine. Ranolazine is used in a dose range from about 1 to about 3000 mg, preferably in a dose range from about 250 to about 2000 mg, and more preferably in a dose range of about 500 to about 1000 mg. Accordingly, the following unit dose combinations of pitavastatin and ranolazine are prepared:

Pitavastatin 1 mg and an extended-release preparation of 500 mg ranolazine;

Pitavastatin 2 mg and an extended-release preparation of 500 mg ranolazine;

Pitavastatin 3 mg and an extended-release preparation of 500 mg ranolazine;

Pitavastatin 4 mg and an extended-release preparation of 500 mg ranolazine;

Pitavastatin 1 mg and an extended-release preparation of 1000 mg ranolazine;

Pitavastatin 2 mg and an extended-release preparation of 1000 mg ranolazine;

Pitavastatin 3 mg and an extended-release preparation of 1000 mg ranolazine;

Pitavastatin 4 mg and an extended-release preparation of 1000 mg ranolazine;

Pitavastatin 1 mg and an extended-release preparation of 2000 mg ranolazine;

Pitavastatin 2 mg and an extended-release preparation of 2000 mg ranolazine;

Pitavastatin 3 mg and an extended-release preparation of 2000 mg ranolazine; and

Pitavastatin 4 mg and an extended-release preparation of 2000 mg ranolazine;

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A method of preventing diabetes resulting from a statin therapy or reducing the risk of diabetes resulting from the statin therapy in a subject who is in need of the statin therapy comprising administering a unit dose combination of HMG-CoA reductase inhibitor and pFOX inhibitor to the subject in an effective therapeutic amount to prevent diabetes resulting from the statin therapy or to reduce the risk of diabetes resulting from the statin therapy.

2. The method of claim 1, wherein the subject newly initiates the statin therapy.

3. The method of claim 1 wherein the HMG-CoA reductase inhibitor is a statin.

4. The method of claim 3 wherein the statin is selected from the group consisting of simvastatin, lovastatin, pravastatin, compactin, fluvastatin, dalvastatin, atorvastatin, rosuvastatin, pitavastatin, and mixtures thereof.

5. The method of claim 3 wherein the statin is simvastatin.

6. The method of claim 3 wherein the statin is lovastatin.

7. The method of claim 3 wherein the statin is pravastatin.

8. The method of claim 3 wherein the statin is fluvastatin.

9. The method of claim 3 wherein the statin is dalvastatin.

10. The method of claim 3 wherein the statin is compactin.

11. The method of claim 3 wherein the statin is atorvastatin.

12. The method of claim 3 wherein the statin is rosuvastatin.

13. The method of claim 3 wherein the statin is pitavastatin.

14. The method of claim 1 wherein the pFOX inhibitor is selected from the group consisting of trimetazidine, ranolazine, CVT 4325, perhexiline, mildronate, etomoxir, dichloroacetate, a pyruvate dehydrogenase agonist, a malonyl CoA decarboxylase inhibitor, and mixtures thereof.

15. The method of claim 3 wherein the statin and pFOX inhibitor are combined in a single tablet, blister pack, administration kit, IV solution, transdermal patch, rectal suppository, inhalational device for immediate, sustained-release or a combination of immediate-release and sustained-release dosing.

16. The method of claim 15 wherein the HMG-CoA reductase inhibitor and pFOX inhibitor are in a single tablet with a dual delivery system which immediately releases the statin and sustained-releases the pFOX inhibitor.

17. The method of claim 16 wherein the statin is atorvastatin and the pFOX inhibitor is trimetazidine.

18. The method of claim 16 wherein the statin is rosuvastatin and the pFOX inhibitor is trimetazidine.

19. The method of claim 16 wherein the statin is pitavastatin and the pFOX inhibitor is trimetazidine.

20. The method of claim 15 wherein the statin is pitavastatin and the pFOX inhibitor is ranolazine.
